(19) United States
(12) Patent Application Publication
(10) Pub. No.: US 2022/0007947 A1
(43) Pub. Date: Jan. 13, 2022

(54) SIGNAL PROCESSING METHOD, APPARATUS AND NON-TRANSITORY COMPUTER READABLE STORAGE MEDIUM

(71) Applicant: Wistron Corporation, New Taipei City (TW)

(72) Inventors: Zheng-De Liu, New Taipei City (TW); Kuo-Ting Huang, New Taipei City (TW)

(73) Assignee: Wistron Corporation, New Taipei City (TW)

(21) Appl. No.: 16/996,923

(22) Filed: Aug. 19, 2020

(30) Foreign Application Priority Data
Jul. 10, 2020 (TW) 109123292

(51) Int. Cl. A61B 5/024; A61B 5/00

(52) U.S. Cl. CPC A61B 5/024; A61B 5/7214; A61B 5/7221; A61B 5/02416

(57) ABSTRACT

A signal processing method, an apparatus and a non-transitory computer readable storage medium are provided. A plurality of segment signals are taken out based on a time series from a physiological signal, and each segment signal is determined as a desirable signal or an undesirable signal. In response to the i^{th} segment signal is determined as the undesirable signal, under the condition of determining that both of the (i−2)^{th} segment signal and the (i+2)^{th} segment signal are the desirable signals, the i^{th} segment signal is reserved. A plurality of instant heart rates are calculated based on the desirable signals and the reserved undesirable signals.

### CROSS-REFERENCE TO RELATED APPLICATION

This application claims the priority benefit of Taiwan application serial no. 109123292, filed on Jul. 10, 2020. The entirety of the above-mentioned patent application is hereby incorporated by reference herein and made a part of this specification.

### BACKGROUND OF THE DISCLOSURE

### 1. Field of the Disclosure

The disclosure relates to a signal processing method, an apparatus and a non-transitory computer readable storage medium, and in particular to a signal processing method, an apparatus and a non-transitory computer readable storage medium for calculating heart rates.

### 2. Description of Related Art

In recent years, with people's emphasis on health, various monitoring devices capable of monitoring heartbeats have appeared on the market. When the wearer is stable, the monitoring device can obtain the best physiological signal. When the wearer has a sudden movement (for example, turns the head, nods, gets up or the like), the monitoring device will produce noise to affect the accuracy. In the past, an acceleration signal of an accelerometer was used to determine whether the wearer had a movement. When the wearer is static, a peak value can be calculated smoothly, then an instant heart rate is calculated from a time difference between the adjacent peak values, and finally, an average heart rate is obtained by making statistics on several heart rates.

When the wearer has a sudden movement (for example, turns the head, nods, gets up or the like), signals sensed by the monitoring device will have noise, so it difficult to calculate the peak value. The current practice is to directly discard signals with noise. However, the instant heart rate is obtained from the adjacent peak values. If a signal is discarded, there will be a lack of two instant heart rates, which will cause a delay in the average heart rate.

### SUMMARY OF THE DISCLOSURE

The disclosure provides a signal processing method, an apparatus and a non-transitory computer readable storage medium, which can find out peak values from a physiological signal of undesirable quality, thereby stably outputting an average heart rate.

The signal processing method of the disclosure includes: taking out a plurality of segment signals based on a time series from a physiological signal; determining each of the segment signals as a desirable signal or an undesirable signal to calculate peak values of these segment signals determined as the desirable signals; in response to the i^{th} segment signal is determined as the undesirable signal, determining whether the (i−2)^{th} segment signal and the (i+2)^{th} segment signal are the desirable signals; under the condition of determining that both of the (i−2)^{th} segment signal and the (i+2)^{th} segment signal are the desirable signals, reserving the i^{th} segment signal determined as the undesirable signal to calculate a representative peak value of the i^{th} segment signal; and calculating a plurality of instant heart rates based on the segment signals determined as the desirable signals and the reserved segment signals determined as the undesirable signals.

In an embodiment of the disclosure, the step of under the condition of determining that both of the (i−2)^{th} segment signal and the (i+2)^{th} segment signal are the desirable signals, reserving the i^{th} segment signal to calculate the representative peak value of the i^{th} segment signal includes: determining a change trend of the i^{th} segment signal based on a first peak value of the (i−2)^{th} segment signal and a second peak value of the (i+2)^{th} segment signal; and obtaining the representative peak value of the i^{th} segment signal by using a calculation manner corresponding to the change trend.

In an embodiment of the disclosure, before the step of determining the change trend of the i^{th} segment signal, the method further includes: calculating an absolute value of a difference between the first peak value and the second peak value. Steps of determining the change trend of the i^{th} segment signal include: comparing the absolute value of the difference with a threshold value; if the absolute value of the difference is less than or equal to the threshold value, determining that the change trend of the i^{th} segment signal is a stable state; in response to the absolute value of the difference is greater than the threshold value and the first peak value is less than the second peak value, determining that the change trend of the i^{th} segment signal is a continuous increase state; and in response to the absolute value of the difference is greater than the threshold value and the first peak value is greater than the second peak value, determining that the change trend of the i^{th} segment signal is a continuous decrease state.

In an embodiment of the disclosure, the step of obtaining the representative peak value of the i^{th} segment signal by using the calculation manner corresponding to the change trend includes: when the change trend is the stable state, obtaining a line segment based on the first peak value and the second peak value; finding out a plurality of candidate peak values from the i^{th} segment signal; and taking out one of the candidate peak values closest to the line segment from these candidate peak values as the representative peak value of the i^{th} segment signal.

In an embodiment of the disclosure, the step of obtaining the representative peak value of the i^{th} segment signal by using the calculation manner corresponding to the change trend includes: under the condition of the change trend is the continuous increase state, obtaining a first line segment based on the first peak value and a third peak value of the (i−1)^{th} segment signal; obtaining a second line segment based on the second peak value and a fourth peak value of the (i+1)^{th} segment signal; finding out a plurality of candidate peak values from the i^{th} segment signal; taking out a first candidate peak value closest to the first line segment from these candidate peak values; taking out a second candidate peak value closest to the second line segment from these candidate peak values; in response to the first candidate peak value and the second candidate peak value are the same, taking the first candidate peak value as the representative peak value of the i^{th} segment signal; and in response to the first candidate peak value and the second candidate peak value are different, taking the smallest of the first candidate peak value and the second candidate peak value as the representative peak value of the i^{th} segment signal.

In an embodiment of the disclosure, the step of obtaining the representative peak value of the i^{th} segment signal by using the calculation manner corresponding to the change trend includes: under the condition of the change trend is the continuous decrease state, obtaining a first line segment based on the first peak value and a third peak value of the (i−1)^{th} segment signal; obtaining a second line segment based on the second peak value and a fourth peak value of the (i+1)^{th} segment signal; finding out a plurality of candidate peak values from the i^{th} segment signal; taking out a first candidate peak value closest to the first line segment from these candidate peak values; taking out a second candidate peak value closest to the second line segment from these candidate peak values; in response to the first candidate peak value and the second candidate peak value are the same, taking the first candidate peak value as the representative peak value of the i^{th} segment signal; and in response to the first candidate peak value and the second candidate peak value are different, taking the largest of the first candidate peak value and the second candidate peak value as the representative peak value of the i^{th} segment signal.

In an embodiment of the disclosure, after determining whether the (i−2)^{th} segment signal and the (i+2)^{th} segment signal are the desirable signals in response to the i^{th} segment signal is determined as the undesirable signal, the method further includes: under the condition of determining that at least one of the (i−2)^{th} segment signal and the (i+2)^{th} segment signal is the undesirable signal, discarding the i^{th} segment signal determined as the undesirable signal.

In an embodiment of the disclosure, the step of determining each of the segment signals as the desirable signal or the undesirable signal includes: determining the segment signals as the desirable signals or the undesirable signals by using a dynamic time warping (DTW) algorithm.

In an embodiment of the disclosure, the signal processing method further includes: after obtaining the representative peak value of the i^{th} segment signal, obtaining a comparison value based on an average value of a first peak value of the (i−2)^{th} segment signal and a second peak value of the (i+2)^{th} segment signal; determining whether the representative peak value is greater than the comparison value; in response to the representative peak value is greater than the comparison value, determining that the i^{th} segment signal is unreliable, and discarding the i^{th} segment signal; and in response to the representative peak value is less than or equal to the comparison value, determining that the i^{th} segment signal is reliable, and reserving the i^{th} segment signal.

The signal processing apparatus of the disclosure includes: a heart rate sensor, sensing a physiological signal of a wearer; a storage unit, including a plurality of code segments; and a processor, coupled to the heart rate sensor and the storage unit. The processor executes the code segments to: receive the physiological signal from the heart rate sensor; take out a plurality of segment signals based on a time series from the physiological signal; determine each of the segment signals as a desirable signal or an undesirable signal to calculate peak values of these segment signals determined as the desirable signals; in response to the i^{th} segment signal is determined as the undesirable signal, determine whether the (i−2)^{th} segment signal and the (i+2)^{th} segment signal are the desirable signals; under the condition of determining that both of the (i−2)^{th} segment signal and the (i+2)^{th} segment signal are the desirable signals, reserve the i^{th} segment signal to calculate a representative peak value of the i^{th} segment signal; when determining that at least one of the (i−2)^{th} segment signal and the (i+2)^{th} segment signal is the undesirable signal, discard the i^{th} segment signal; and calculate a plurality of instant heart rates based on the segment signals determined as the desirable signals and the reserved segment signals determined as the undesirable signals.

The non-transitory computer readable storage medium of the disclosure records at least one programmed instruction. After being loaded into an electronic device, the at least one programmed instruction performs the following steps: taking out a plurality of segment signals based on a time series from a physiological signal; determining each of the segment signals as a desirable signal or an undesirable signal to calculate peak values of these segment signals determined as the desirable signals; in response to the i^{th} segment signal is determined as the undesirable signal, determining whether the (i−2)^{th} segment signal and the (i+2)^{th} segment signal are the desirable signals; when determining that both of the (i−2)^{th} segment signal and the (i+2)^{th} segment signal are the desirable signals, reserving the i^{th} segment signal to calculate a representative peak value of the i^{th} segment signal; under the condition of determining that at least one of the (i−2)^{th} segment signal and the (i+2)^{th} segment signal is the undesirable signal, discarding the i^{th} segment signal; and calculating a plurality of instant heart rates based on the segment signals determined as the desirable signals and the reserved segment signals determined as the undesirable signals.

Based on the above, the disclosure can further determine whether to reserve the undesirable signals to find out the peak values from the undesirable signals, thereby stably outputting the average heart rate.

### BRIEF DESCRIPTION OF THE DRAWINGS

FIG. 1 is a block diagram of a signal processing apparatus according to an embodiment of the disclosure.

FIG. 2 is a flow chart of a signal processing method according to an embodiment of the disclosure.

FIG. 3A is a schematic diagram of a physiological signal according to an embodiment of the disclosure.

FIG. 3B is a schematic diagram of calculating instant heart rates according to an embodiment of the disclosure.

FIG. 4 is a waveform diagram of a stable state according to an embodiment of the disclosure.

FIG. 5 is a waveform diagram of a continuous increase state according to an embodiment of the disclosure.

FIG. 6 is a waveform diagram of a continuous decrease state according to an embodiment of the disclosure.

FIG. 7 is a waveform diagram of a calculation manner of a representative peak value in a stable state according to an embodiment of the disclosure.

FIG. 8A to FIG. 8B are waveform diagrams of a calculation manner of a representative peak value in a continuous increase state according to an embodiment of the disclosure.

FIG. 9A to FIG. 9B are waveform diagrams of a calculation manner of a representative peak value in a continuous decrease state according to an embodiment of the disclosure.

### DESCRIPTION OF THE EMBODIMENTS

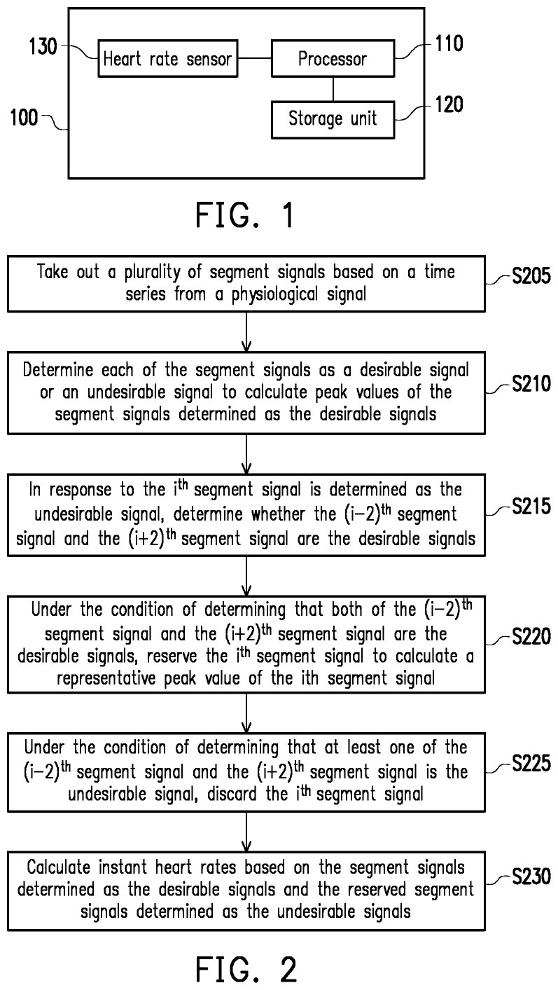

FIG. 1 is a block diagram of a signal processing apparatus according to an embodiment of the disclosure. Referring to FIG. 1, the signal processing apparatus 100 includes a processor 110, a storage unit 120 and a heart rate sensor 130. The processor 110 is directly or indirectly electrically coupled to the storage unit 120 and the heart rate sensor 130.

The processor 110 is, for example, a central processing unit (CPU), a physical processing unit (PPU), a programmable microprocessor (microprocessor), an embedded control chip, a digital signal processor (DSP), an application specific integrated circuit (ASIC) or other similar devices.

The storage unit 120 is, for example, any type of fixed or movable random access memory (RAM), read-only memory (ROM), flash memory, hard disk, or other similar devices or a combination of these devices. A plurality of code segments are stored in the storage unit 120. After the above code segments are installed, the processor 110 executes the code segments to realize the following signal processing method.

The heart rate sensor 130 is configured to sense a physiological signal of a wearer. The heart rate sensor 130 is, for example, a sensor using photoplethysmography (PPG), but is not limited thereto, and may be, for example, a radar sensor or an electrocardiogram (ECG) sensor.

FIG. 2 is a flow chart of a signal processing method according to an embodiment of the disclosure. In the embodiment, the processor 110 is configured to execute the code segments stored in the storage unit 120, thereby realizing the following signal processing method.

In step S205, a plurality of segment signals are taken out based on a time series from a physiological signal. The processor 110 collects the physiological signal within a sampling time range through the heart rate sensor 130, and divides the physiological signal into the plurality of segment signals. Next, in step S210, each of the segment signals is determined as a desirable signal or an undesirable signal to calculate peak values of the segment signals determined as the desirable signals. Moreover, it is further determined to reserve or discard the undesirable signals and calculate a representative peak value of the reserved undesirable signals.

The processor 110 captures a fixed-length signal by using a sampling window to calculate a peak value here. After the peak value in the sampling window is calculated, a peak value of a signal in the next sampling window is calculated after translating the sampling window back a distance based on the time series. The above translation distance is, for example, ½ a width of the sampling window.

When the wearer is stable and has no movement, the physiological signal detected by the heart rate sensor 130 has obvious and regular peak values. When the wearer has a movement, the physiological signal detected by the heart rate sensor 130 has noise. When the physiological signal is mixed with noise, it is difficult to determine a desirable signal, and an intensity of the noise may be higher than the desirable signal, so the intensity cannot be used to determine the desirable signal. The undesirable signal may be defined as having too many peak values here. For example, when the number of the peak values in the segment signal is greater than 1, the segment signal can be determined as the undesirable signal.

In addition, the processor 110 may determine the segment signals as the desirable signals or the undesirable signals by using a dynamic time warping (DTW) algorithm. A principle of the DTW algorithm is to provide a sample waveform and determine whether each waveform to be tested matches the sample waveform.

Therefore, a sample waveform of the desirable signal is provided here, so that the waveform of each of the segment signals (that is, the waveform to be tested) is compared with the sample waveform to determine each of the segment signals as the desirable signal or the undesirable signal.

For the i^{th} segment signal determined as the undesirable signal, the processor 110 may further determine a change trend according to the adjacent segment signals in the time series, thereby calculating a representative peak value of the i^{th} segment signal. Thus, it is necessary to firstly determine whether the adjacent segment signals in the time series are reliable, that is, whether they are desirable signals.

Since the physiological signal is a continuous signal, the signal will conform to an overall change trend, so that the peak value close to the overall trend is more likely to be an actual signal, and the peak value that deviates from the overall trend is more likely to be noise. Therefore, in the embodiment, the second segment signal ahead and the second segment signal behind (the (i−2)^{th} segment signal and the (i+2)^{th} segment signal) of the undesirable signal (i^{th} segment signal) are particularly taken out to determine the change trend. The reason for not taking the adjacent previous segment signal and the next segment signal (the (i−1)^{th} segment signal and the (i+1)^{th} segment signal) is that the peak values of the adjacent segment signals are close to the undesirable signal, and may be unstable peak values or be affected by the undesirable signal. Therefore, the peak values of the second segment signal ahead and the second segment signal behind (the (i−2)^{th} segment signal and the (i+2)^{th} segment signal) of the undesirable signal are taken out to further calculate the representative peak value of the undesirable signal. On the one hand, the change trend is still reserved, and on the other hand, it is not easily affected by the undesirable signal.

In the embodiment, the method of calculating the peak value is real-time calculation. Therefore, when the segment signal is determined as the undesirable signal, the segment signal is firstly temporarily stored, and after the peak values of the subsequent two segment signals are taken, it can be determined to reserve or discard the undesirable signal. For the determination to reserve or discard the undesirable signal, reference may be made to step S215 to step S225. In addition, for example, a flag “true” is given to each of the reserved segment signals, and a flag “false” is given to each of the discarded segment signals.

In step S215, if the i^{th} segment signal is determined as the undesirable signal, it is determined whether the (i−2)^{th} segment signal and the (i+2)^{th} segment signal are desirable signals. In step S220, when determining that both of the (i−2)^{th} segment signal and the (i+2)^{th} segment signal are the desirable signals, the i^{th} segment signal is reserved to calculate a representative peak value of the i^{th} segment signal. In step S225, when determining that at least one of the (i−2)^{th} segment signal and the (i+2)^{th} segment signal is the undesirable signal, the i^{th} segment signal is discarded.

For example, assuming that the 3^{rd} segment signal is determined as an undesirable signal, the processor 110 will firstly temporarily store the 3^{rd} segment signal to wait for the obtainment of the 5^{th} segment signal. The processor 110 further determines whether both the 1^{st} segment signal and the 5^{th} whether are the desirable signals. If both of the 1^{st} segment signal and the 5^{th} segment signal are the desirable signals, the 3^{rd} segment signal is reserved, and a representative peak value of the 3^{rd} segment signal is further calculated. If one of the 1^{st} segment signal and the 5^{th} segment signal is the undesirable signal, the 3^{rd} segment signal is discarded, that is, the representative peak value of the 3^{rd} segment signal is not calculated.

Finally, in step S230, instant heart rates are calculated based on the segment signals determined as the desirable signals and the reserved segment signals determined as the undesirable signals. The processor 110 calculates the instant heart rate by using a time difference between the 2 adjacent peak values here, and finally makes statistics on several instant heart rates to obtain an average heart rate.

Generally speaking, the heartbeats per minute F (instant heart rate) is obtained by calculating a time difference T between two adjacent peak values (that is, a heart rate cycle) and then calculating a reciprocal thereof. F=1/T (heartbeats per second)=60/T (heartbeats per minute).

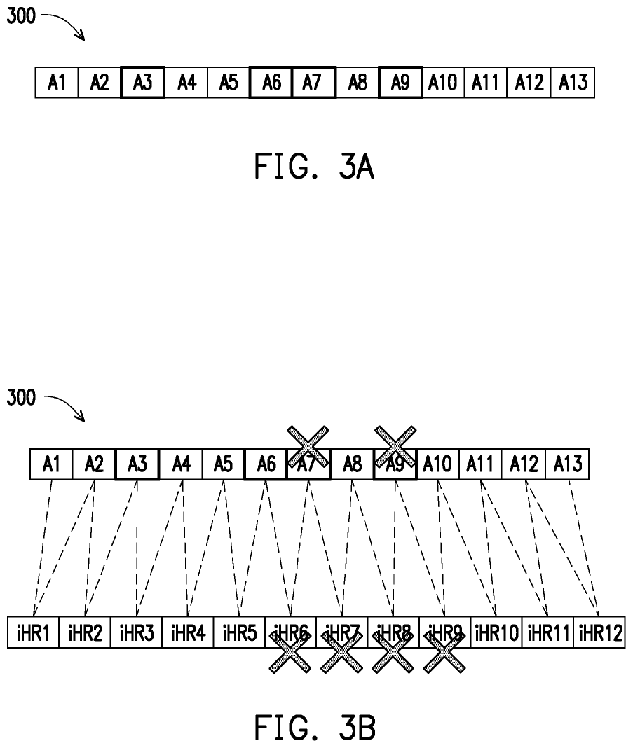

FIG. 3A is a schematic diagram of a physiological signal according to an embodiment of the disclosure. FIG. 3B is a schematic diagram of calculating instant heart rates according to an embodiment of the disclosure. Referring to FIG. 3A, a physiological signal 300 includes a plurality of segment signals, and 13 segment signals A1 to A13 are shown here. The segment signals A1, A2, A4, A5, A8 and A10 to A13 represent desirable signals, and the segment signals A3, A6, A7 and A9 represent undesirable signals. In terms of the segment signal A3, the segment signal A1 is taken ahead and the segment signal A5 is taken behind. Both of the segment signal A1 and the segment signal A5 are the desirable signals, so the segment signal A3 determined as the undesirable signal is reserved. By analogy, the segment signals A3 and A6 are reserved, and the segment signals A7 and A9 are discarded.

Referring to FIG. 3B, the processor 110 obtains instant heart rates based on a time difference of peak values of two adjacent time segments. Based on the above embodiment, the segment signals A3 and A6 determined as the undesirable signals are reserved, so instant heart rates iHR1 to iHR5 may be obtained based on the time segments A1 to A6. Instant heart rates iHR10 to iHR12 may be obtained based on the time segments A10 to A13. In addition, since the time segments A7 and A9 have been discarded, instant heart rates iHR6 to iHR9 that could have been obtained based on the time segments A6 to A10 cannot be obtained. The processor 110 may calculate an average heart rate after obtaining a preset number of instant heart rates here. For example, it may be designed to calculate the average heart rate with 5 instant heart rates. Therefore, through the method of the embodiment, 5 instant heart rates iHR1 to iHR5 may be obtained based on the time segments A1 to A6. Average heart rate=(iHR1+iHR2+iHR3+iHR4+iHR5)/5.

If the method of the above embodiment is not used, since all of the segment signals A3, A6, A7 and A8 are determined as the undesirable signals, all of the instant heart rates iHR2, iHR3 and iHR5 to iHR9 are unavailable, and it is necessary to delay to the segment signal A13 to obtain 5 instant heart rates iHR1, iHR4, iHR10, iHR11 and iHR12. Therefore, through the method of the above embodiment, the problem of delay in the calculation of the average heart rate can be solved.

In addition, in the above step S220, the representative peak value of the i^{th} segment signal may be calculated based on a first peak value of the (i−2)^{th} segment signal and a second peak value of the (i+2)^{th} segment signal, and may be selected from a plurality of candidate peak values included in the i^{th} segment signal. Specifically, the processor 110 determines a change trend of the i^{th} segment signal based on the first peak value of the (i−2)^{th} segment signal and the second peak value of the (i+2)^{th} segment signal. Then, the processor 110 obtains the representative peak value of the i^{th} segment signal by using a calculation manner corresponding to the change trend.

The i^{th} segment signal determined as the undesirable signal has three change trends, including a stable state, a continuous increase state and a continuous decrease state. The processor 110 calculates an absolute value of a difference between the first peak value and the second peak value (|first peak value-second peak value|), and determines the change trend of the i^{th} segment signal based on the absolute value of the difference. The processor 110 compares the absolute value of the difference with a preset threshold value here. If the absolute value of the difference is less than or equal to the threshold value, it is determined that the change trend of the i^{th} segment signal is the stable state. If the absolute value of the difference is greater than the threshold value and the first peak value is less than the second peak value, it is determined that the change trend of the i^{th} segment signal is the continuous increase state. If the absolute value of the difference is greater than the threshold value and the first peak value is greater than the second peak value, it is determined that the change trend of the i^{th} segment signal is the continuous decrease state.

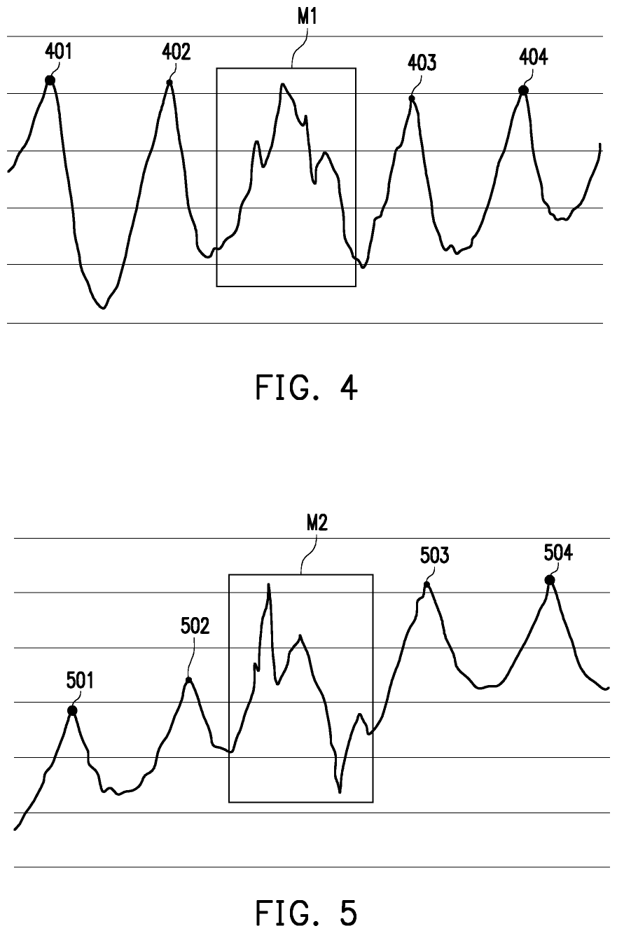

The stable state, the continuous increase state and the continuous decrease state are respectively described below with reference to FIG. 4 to FIG. 6.

FIG. 4 is a waveform diagram of a stable state according to an embodiment of the disclosure. In FIG. 4, it is assumed that the m^{th} segment signal to the (m+4)^{th} segment signal are shown. The (m+2)^{th} segment signal (segment signal M1) is an undesirable signal, and the other segment signals are desirable signals. Peak values 401, 402, 403 and 404 are respectively peak values of the m^{th} segment signal, the (m+1)^{th} segment signal, the (m+3)^{th} segment signal and the (m+4)^{th} segment signal.

In terms of the segment signal M1, the processor 110 takes the peak value 401 (first peak value) of the m^{th} segment signal ahead, takes the peak value 404 (second peak value) of the (m+4)^{th} segment signal behind, calculates an absolute value of a difference between the peak value 401 and the peak value 404, and compares the absolute value of the difference with a preset threshold value. Since the absolute value of the difference between the peak value 401 and the peak value 404 is not greater than the threshold value, it is determined that the change trend of the segment signal M1 is the stable state. It is assumed that the preset threshold value is 1500 millivolts (mV) here.

FIG. 5 is a waveform diagram of a continuous increase state according to an embodiment of the disclosure. In FIG. 5, it is assumed that the n^{th} segment signal to the (n+4)^{th} segment signal are shown. The (n+2)^{th} segment signal (segment signal M2) is an undesirable signal, and the other segment signals are desirable signals. Peak values 501, 502, 503 and 504 are respectively peak values of the n^{th} segment signal, the (n+1)^{th} segment signal, the (n+3)^{th} segment signal and the (n+4)^{th} segment signal.

In terms of the segment signal M2, the processor 110 takes the peak value 501 (first peak value) of the n^{th} segment signal ahead, takes the peak value 504 (second peak value) of the (n+4)^{th} segment signal behind, calculates an absolute value of a difference between the peak value 501 and the peak value 504, and compares the absolute value of the difference with a preset threshold value. Since the absolute value of the difference between the peak value 501 and the peak value 504 is greater than the threshold value, it is further determined whether the peak value 501 or the peak value 504 is greater. The peak value 504 is greater than the peak value 501 here, so it is determined that the change trend of the segment signal M2 is the continuous increase state.

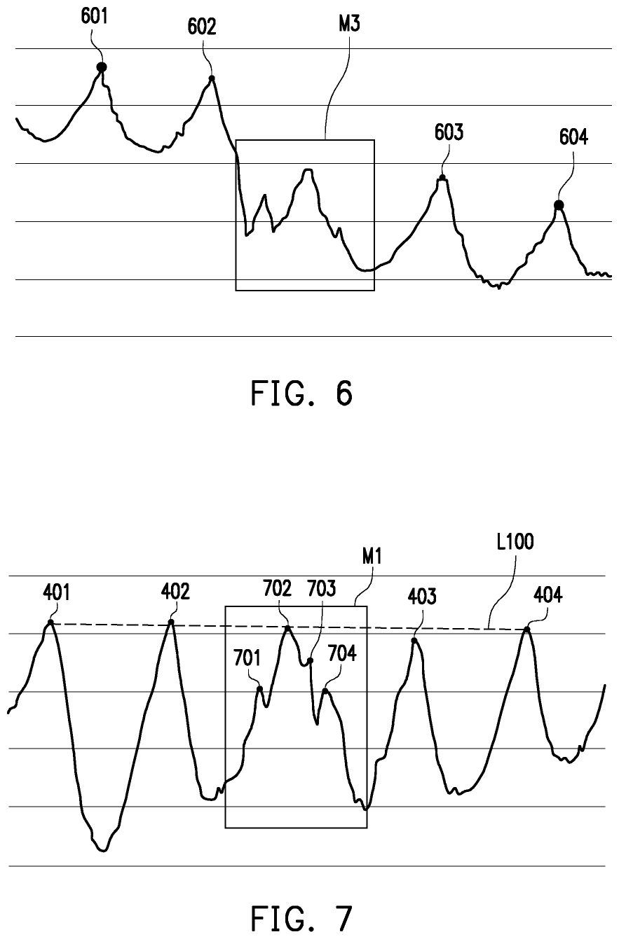

FIG. 6 is a waveform diagram of a continuous decrease state according to an embodiment of the disclosure. In FIG. 6, it is assumed that the p^{th} segment signal to the (p+4)^{th} segment signal are shown. The (p+2)^{th} segment signal (segment signal M3) is an undesirable signal, and the other segment signals are desirable signals. Peak values 601, 602, 603 and 604 are respectively peak values of the p^{th} segment signal, the (p+1)^{th} segment signal, the (p+3)^{th} segment signal and the (p+4)^{th} segment signal.

In terms of the segment signal M3, the processor 110 takes the peak value 601 (first peak value) of the p^{th} segment signal ahead, takes the peak value 604 (second peak value) of the (p+4)^{th} segment signal behind, calculates an absolute value of a difference between the peak value 601 and the peak value 604, and compares the absolute value of the difference with a preset threshold value. Since the absolute value of the difference between the peak value 601 and the peak value 604 is greater than the threshold value, it is further determined whether the peak value 601 or the peak value 604 is greater. The peak value 601 is greater than the peak value 604 here, so it is determined that the change trend of the segment signal M3 is the continuous decrease state.

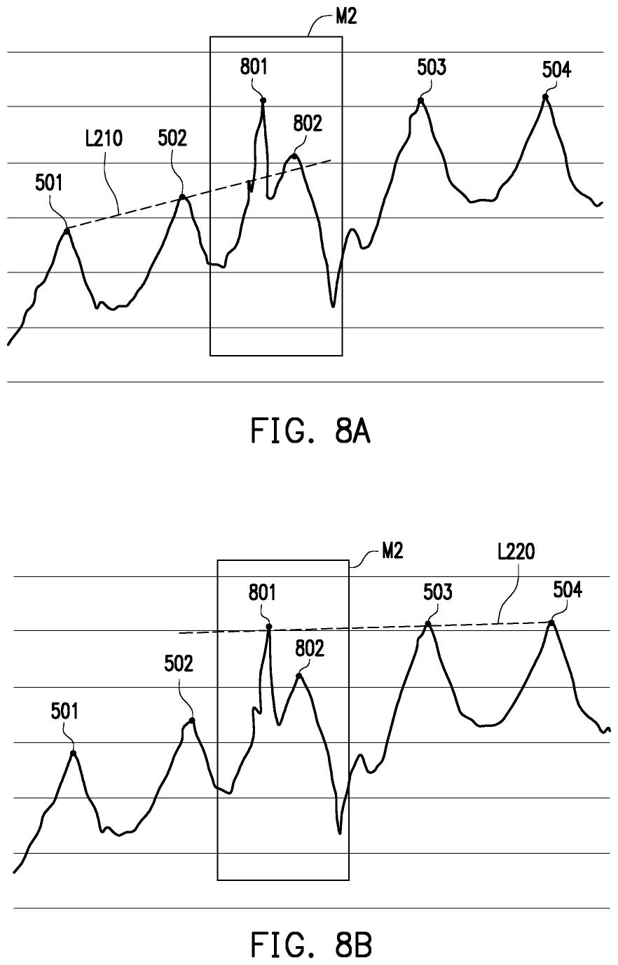

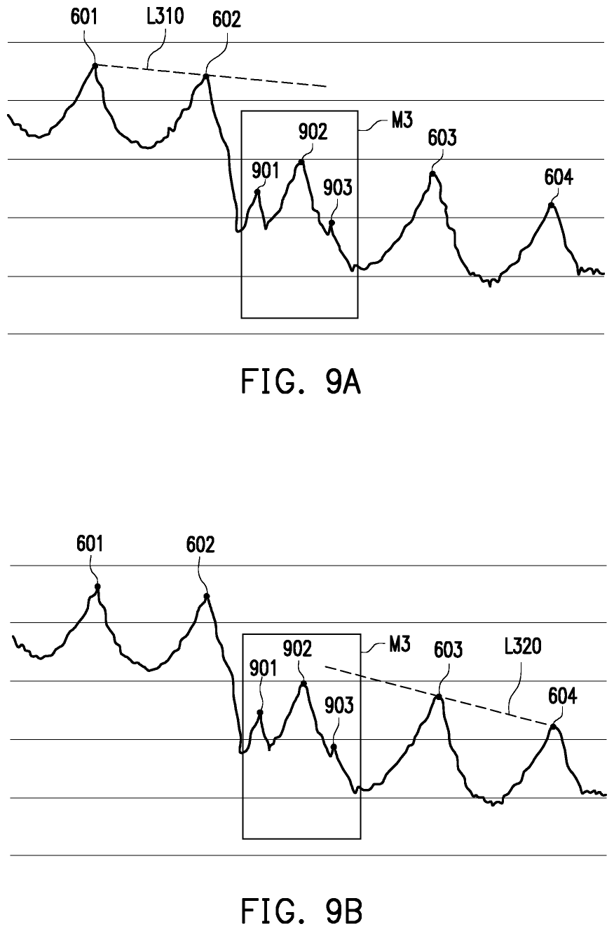

After obtaining the change trend of the undesirable signal, the processor 110 may obtain the representative peak value of the undesirable signal by using the calculation manner corresponding to the change trend. The calculation manners of the representative peak values in the stable state, the continuous increase state and the continuous decrease state are respectively described below with reference to FIG. 7, FIG. 8A to FIG. 8B and FIG. 9A to FIG. 9B.

FIG. 7 is a waveform diagram of a calculation manner of a representative peak value in a stable state according to an embodiment of the disclosure. Here, the embodiment of FIG. 4 is used to describe the calculation manner of the representative peak value in the stable state.

Referring to FIG. 7, firstly, the processor 110 obtains a line segment L100 based on the peak value 401 (first peak value) and the peak value 404 (second peak value). Next, the processor 110 finds out a plurality of candidate peak values 701 to 704 from the segment signal M1, and takes out one of the candidate peak values closest to the line segment L100 from these candidate peak values 701 to 704 as a representative peak value of the segment signal M1. The candidate peak value 702 is selected as the representative peak value of the segment signal M1.

FIG. 8A to FIG. 8B are waveform diagrams of a calculation manner of a representative peak value in a continuous increase state according to an embodiment of the disclosure. The embodiment of FIG. 5 is used to describe the calculation manner of the representative peak value in the continuous increase state here.

Referring to FIG. 8A to FIG. 8B, the (n+2)^{th} segment signal (segment signal M2) is the undesirable signal. The processor 110 obtains a line segment L210 based on the peak value 501 (first peak value) of the n^{th} segment signal and the peak value 502 (third peak value) of the (n+1)^{th} segment signal. Moreover, the processor 110 obtains a line segment L220 based on the peak value 504 (second peak value) of the (n+4)^{th} segment signal and the peak value 503 (fourth peak value) of the (n+3)^{th} segment signal.

Moreover, the processor 110 finds out a plurality of candidate peak values 801 and 802 from the segment signal M2. Next, the processor 110 takes out the candidate peak value 802 (first candidate peak value) closest to the line segment L210 from these candidate peak values 801 and 802, and takes out the candidate peak value 801 (second candidate peak value) closest to the line segment L220 from these candidate peak values 801 and 802. Next, the processor 110 determines whether the first candidate peak value and the second candidate peak value are the same.

In the waveform where the change trend is the continuous increase state, since a surge with a sudden increase in intensity is more likely to be noise, if the first candidate peak value and the second candidate peak value are different, the smallest of the first candidate peak value and the second candidate peak value is taken as the representative peak value of the segment signal M2. In the embodiment of FIG. 8A to FIG. 8B, the first candidate peak value (candidate peak value 802) and the second candidate peak value (candidate peak value 801) are different, so the processor 110 selects the smallest candidate peak value 802 of the two as the representative peak value of the segment signal M2.

In other embodiments, if the first candidate peak value closest to the line segment L210 and the second candidate peak value closest to the line segment L220 are the same, the processor 110 takes the first candidate peak value (that is, the second candidate peak value) as the representative peak value of the segment signal M2.

FIG. 9A to FIG. 9B are waveform diagrams of a calculation manner of a representative peak value in a continuous decrease state according to an embodiment of the disclosure. The embodiment of FIG. 6 is used to describe the calculation manner of the representative peak value in the continuous decrease state here.

Referring to FIG. 9A to FIG. 9B, the (p+2)^{th} segment signal (segment signal M3) is the undesirable signal. The processor 110 obtains a line segment L310 based on the peak value 601 (first peak value) of the p^{th} segment signal and the peak value 602 (third peak value) of the (p+1)^{th} segment signal. Moreover, the processor 110 obtains a line segment L320 based on the peak value 604 (second peak value) of the (p+4)^{th} segment signal and the peak value 603 (fourth peak value) of the (p+3)^{th} segment signal.

Moreover, the processor 110 finds out a plurality of candidate peak values 901, 902 and 903 from the segment signal M3. Next, the processor 110 takes out the candidate peak value 902 (first candidate peak value) closest to the line segment L310 from these candidate peak values 901, 902 and 903, and takes out the candidate peak value 902 (second candidate peak value) closest to the line segment L320 from these candidate peak values 901, 902 and 903. Next, the processor 110 determines whether the first candidate peak value and the second candidate peak value are the same.

In the embodiment, both of the first candidate peak value and the second candidate peak value are the candidate peak value 902, so the processor 110 takes the candidate peak value 902 as the representative peak value of the segment signal M3.

In other embodiments, in the waveform where the change trend is the continuous decrease state, since a surge with a sudden decrease in intensity is more likely to be noise, if the first candidate peak value closest to the line segment L310 and the second candidate peak value closest to the line segment L320 are different, the largest of the first candidate peak value and the second candidate peak value is taken as the representative peak value of the segment signal M3.

In addition, in the above embodiment, the representative peak value of the undesirable signal is further calculated. Therefore, in order to avoid a too absurd determination result, after obtaining the representative peak value of the undesirable signal, it is further calculated whether a voltage value of the representative peak value of the undesirable signal is too high. For example, the i^{th} segment signal is determined as the undesirable signal, and a comparison value is obtained based on an average value of a first peak value of the (i−2)^{th} segment signal and a second peak value of the (i+2)^{th} segment signal. Comparison value=2×ACAVG(S1AC:S2AC). ACAVG(S1AC:S2AC) represents an average value of a voltage value S1AC of the first peak value of the (i−2)^{th} segment signal and a voltage value S2AC of the second peak value of the (i+2)^{th} segment signal.

Next, the representative peak value of the undesirable signal is compared with the comparison value. If the representative peak value is greater than the comparison value, it is determined that the undesirable signal is unreliable, and the undesirable signal is discarded. If the representative peak value is less than or equal to the comparison value, it is determined that the undesirable signal is reliable, and the undesirable signal is reserved.

In addition, the above embodiments may be realized by a non-transitory computer readable storage medium. The non-transitory computer readable storage medium records at least one programmed instruction. After being loaded into an electronic device, the programmed instruction performs steps S205 to S230.

Based on the above, in the disclosure, for the segment signal of undesirable quality, the possible representative peak value is calculated based on the change trend of the signals ahead and behind, the peak values are found out from the PPG signal of undesirable quality, the problem of delay in the average heart rate caused by the undesirable quality of the physiological signal is solved, and the average heart rate can be output stably.

What is claimed is:

1. A signal processing method, comprising:
taking out a plurality of segment signals based on a time series from a physiological signal;
determining each of the plurality of segment signals as a desirable signal or an undesirable signal to calculate peak values of the plurality of segment signals determined as the desirable signals;
in response to a i^{th} segment signal is determined as the undesirable signal, determining whether a (i−2)^{th} segment signal and a (i+2)^{th} segment signal are the desirable signals;
under the condition of determining that both of the (i−2)^{th} segment signal and the (i+2)^{th} segment signal are the desirable signals, reserving the i^{th} segment signal determined as the undesirable signal to calculate a representative peak value of the i^{th} segment signal; and
calculating a plurality of instant heart rates based on the segment signals determined as the desirable signals and the reserved segment signals determined as the undesirable signals.

2. The signal processing method according to claim 1, wherein the step of reserving the i^{th} segment signal to calculate the representative peak value of the i^{th} segment signal under the condition of determining that both of the (i−2)^{th} segment signal and the (i+2)^{th} segment signal are the desirable signals comprises:
determining a change trend of the i^{th} segment signal based on a first peak value of the (i−2)^{th} segment signal and a second peak value of the (i+2)^{th} segment signal; and
obtaining the representative peak value of the i^{th} segment signal by using a calculation manner corresponding to the change trend.

3. The signal processing method according to claim 2, wherein before the step of determining the change trend of the i^{th} segment signal, the method further comprises:
calculating an absolute value of a difference between the first peak value and the second peak value;
steps of determining the change trend of the i^{th} segment signal comprise:
comparing the absolute value of the difference with a threshold value;
in response to the absolute value of the difference is less than or equal to the threshold value, determining that the change trend of the i^{th} segment signal is a stable state;
in response to the absolute value of the difference is greater than the threshold value and the first peak value is less than the second peak value, determining that the change trend of the i^{th} segment signal is a continuous increase state; and
in response to the absolute value of the difference is greater than the threshold value and the first peak value is greater than the second peak value, determining that the change trend of the i^{th} segment signal is a continuous decrease state.

4. The signal processing method according to claim 3, wherein the step of obtaining the representative peak value of the i^{th} segment signal by using the calculation manner corresponding to the change trend comprises:
under the condition that the change trend is the stable state,
obtaining a line segment based on the first peak value and the second peak value;
finding out a plurality of candidate peak values from the i^{th} segment signal; and
taking out one of the candidate peak values closest to the line segment from the plurality of candidate peak values as the representative peak value of the i^{th} segment signal.

5. The signal processing method according to claim 3, wherein the step of obtaining the representative peak value of the i^{th} segment signal by using the calculation manner corresponding to the change trend comprises:
under the condition that the change trend is the continuous increase state,
obtaining a first line segment based on the first peak value and a third peak value of a (i−1)^{th} segment signal;
obtaining a second line segment based on the second peak value and a fourth peak value of a (i+1)^{th} segment signal;
finding out a plurality of candidate peak values from the i^{th} segment signal;
taking out a first candidate peak value closest to the first line segment from the plurality of candidate peak values;
taking out a second candidate peak value closest to the second line segment from the plurality of candidate peak values;
in response to the first candidate peak value and the second candidate peak value are the same, taking the first candidate peak value as the representative peak value of the i^{th} segment signal; and
in response to the first candidate peak value and the second candidate peak value are different, taking the smallest of the first candidate peak value and the second candidate peak value as the representative peak value of the i^{th} segment signal.

6. The signal processing method according to claim 3, wherein the step of obtaining the representative peak value of the i^{th} segment signal by using the calculation manner corresponding to the change trend comprises:
under the condition that the change trend is the continuous decrease state,
obtaining a first line segment based on the first peak value and a third peak value of a (i−1)^{th} segment signal;
obtaining a second line segment based on the second peak value and a fourth peak value of a (i+1)^{th} segment signal;
finding out a plurality of candidate peak values from the i^{th} segment signal;
taking out a first candidate peak value closest to the first line segment from the plurality of candidate peak values;
taking out a second candidate peak value closest to the second line segment from the plurality of candidate peak values;
in response to the first candidate peak value and the second candidate peak value are the same, taking the first candidate peak value as the representative peak value of the i^{th} segment signal; and
in response to the first candidate peak value and the second candidate peak value are different, taking the largest of the first candidate peak value and the second candidate peak value as the representative peak value of the i^{th} segment signal.

7. The signal processing method according to claim 1, wherein after the step of determining whether the (i−2)^{th} segment signal and the (i+2)^{th} segment signal are the desirable signals in response to the i^{th} segment signal is determined as the undesirable signal, the method further comprises:
under the condition of determining that at least one of the (i−2)^{th} segment signal and the (i+2)^{th} segment signal is the undesirable signal, discarding the i^{th} segment signal determined as the undesirable signal.

8. The signal processing method according to claim 1, wherein the step of determining each of the plurality of segment signals as the desirable signal or the undesirable signal comprises:
determining each of the plurality of segment signals as the desirable signal or the undesirable signal by using a dynamic time warping algorithm.

9. The signal processing method according to claim 1, further comprising:
after obtaining the representative peak value of the i^{th} segment signal, obtaining a comparison value based on an average value of a first peak value of the (i−2)^{th} segment signal and a second peak value of the (i+2)^{th} segment signal;
determining whether the representative peak value is greater than the comparison value;
in response to the representative peak value is greater than the comparison value, determining that the i^{th} segment signal is unreliable, and discarding the i^{th} segment signal; and
in response to the representative peak value is less than or equal to the comparison value, determining that the i^{th} segment signal is reliable, and reserving the i^{th} segment signal.

10. A signal processing apparatus, comprising:
a heart rate sensor, sensing a physiological signal of a wearer;
a storage unit, comprising a plurality of code segments; and
a processor, coupled to the heart rate sensor and the storage unit, wherein the processor executes the plurality of code segments to:
receive the physiological signal from the heart rate sensor;
take out a plurality of segment signals based on a time series from the physiological signal;
determine each of the plurality of segment signals as a desirable signal or an undesirable signal to calculate peak values of the plurality of segment signals determined as the desirable signals;
in response to the i^{th} segment signal is determined as the undesirable signal, determine whether the (i−2)^{th} segment signal and the (i+2)^{th} segment signal are the desirable signals;
under the condition of determining that both of the (i−2)^{th} segment signal and the (i+2)^{th} segment signal are the desirable signals, reserve the i^{th} segment signal determined as the undesirable signal to calculate a representative peak value of the i^{th} segment signal; and
calculate a plurality of instant heart rates based on the segment signals determined as the desirable signals and the reserved segment signals determined as the undesirable signals.

11. The signal processing apparatus according to claim 10, wherein the processor executes the plurality of code segments to:
determine a change trend of the i^{th} segment signal based on a first peak value of the (i−2)^{th} segment signal and a second peak value of the (i+2)^{th} segment signal; and
obtain the representative peak value of the i^{th} segment signal by using a calculation manner corresponding to the change trend.

12. The signal processing apparatus according to claim 10, wherein the processor executes the plurality of code segments to:
before determining a change trend of the i^{th} segment signal, calculate an absolute value of a difference between a first peak value and a second peak value;
compare the absolute value of the difference with a threshold value, thereby determining the change trend of the i^{th} segment signal;
in response to the absolute value of the difference is less than or equal to the threshold value, determine that the change trend of the i^{th} segment signal is a stable state;
in response to the absolute value of the difference is greater than the threshold value and the first peak value is less than the second peak value, determine that the change trend of the i^{th} segment signal is a continuous increase state; and
in response to the absolute value of the difference is greater than the threshold value and the first peak value is greater than the second peak value, determine that the change trend of the i^{th} segment signal is a continuous decrease state.

13. The signal processing apparatus according to claim 12, wherein the processor executes the plurality of code segments to:
under the condition that the change trend is the stable state,
obtain a line segment based on the first peak value and the second peak value;
find out a plurality of candidate peak values from the i^{th} segment signal; and
take out one of the candidate peak values closest to the line segment from the plurality of candidate peak values as the representative peak value of the i^{th} segment signal.

14. The signal processing apparatus according to claim 12, wherein the processor executes the plurality of code segments to:
under the condition that the change trend is the continuous increase state,
obtain a first line segment based on the first peak value and a third peak value of a (i−1)^{th} segment signal;
obtain a second line segment based on the second peak value and a fourth peak value of a (i+1)^{th} segment signal;
find out a plurality of candidate peak values from the i^{th} segment signal;
take out a first candidate peak value closest to the first line segment from the plurality of candidate peak values;
take out a second candidate peak value closest to the second line segment from the plurality of candidate peak values;
in response to the first candidate peak value and the second candidate peak value are the same, take the first candidate peak value as the representative peak value of the i^{th} segment signal; and
in response to the first candidate peak value and the second candidate peak value are different, take the smallest of the first candidate peak value and the second candidate peak value as the representative peak value of the i^{th} segment signal.

15. The signal processing apparatus according to claim 12, wherein the processor executes the plurality of code segments to:
under the condition that the change trend is the continuous decrease state,
obtain a first line segment based on the first peak value and a third peak value of a (i−1)^{th} segment signal;
obtain a second line segment based on the second peak value and a fourth peak value of a (i+1)^{th} segment signal;
find out a plurality of candidate peak values from the i^{th} segment signal;
take out a first candidate peak value closest to the first line segment from the plurality of candidate peak values;
take out a second candidate peak value closest to the second line segment from the plurality of candidate peak values;
in response to the first candidate peak value and the second candidate peak value are the same, take the first candidate peak value as the representative peak value of the i^{th} segment signal; and
in response to the first candidate peak value and the second candidate peak value are different, take the largest of the first candidate peak value and the second candidate peak value as the representative peak value of the i^{th} segment signal.

16. The signal processing apparatus according to claim 10, wherein the processor executes the plurality of code segments to:
in response to the i^{th} segment signal is determined as the undesirable signal, under the condition of determining that at least one of the (i−2)^{th} segment signal and the (i+2)^{th} segment signal is the undesirable signal, discard the i^{th} segment signal determined as the undesirable signal.

17. The signal processing apparatus according to claim 10, wherein the processor executes the plurality of code segments to:
determine each of the plurality of segment signals as the desirable signal or the undesirable signal by using a dynamic time warping algorithm.

18. The signal processing apparatus according to claim 10, wherein the processor executes the plurality of code segments to:
after obtaining the representative peak value of the i^{th} segment signal, obtain a comparison value based on an average value of a first peak value of the (i−2)^{th} segment signal and a second peak value of the (i+2)^{th} segment signal;
determine whether the representative peak value is greater than the comparison value;
in response to the representative peak value is greater than the comparison value, determine that the i^{th} segment signal is unreliable, and discard the i^{th} segment signal; and
in response to the representative peak value is less than or equal to the comparison value, determine that the i^{th} segment signal is reliable, and reserve the i^{th} segment signal.

19. A non-transitory computer readable storage medium, wherein the non-transitory computer readable storage medium records at least one programmed instruction, and after being loaded into an electronic device, the at least one programmed instruction performs the following steps:
taking out a plurality of segment signals based on a time series from a physiological signal;
determining each of the plurality of segment signals as a desirable signal or an undesirable signal to calculate peak values of the plurality of segment signals determined as the desirable signals;
in response to the i^{th} segment signal is determined as the undesirable signal, determining whether the (i−2)^{th} segment signal and the (i+2)^{th} segment signal are the desirable signals;
under the condition of determining that both of the (i−2)^{th} segment signal and the (i+2)^{th} segment signal are the desirable signals, reserving the i^{th} segment signal to calculate a representative peak value of the i^{th} segment signal; and
calculating a plurality of instant heart rates based on the segment signals determined as the desirable signals and the reserved segment signals determined as the undesirable signals.
